(19) United States
(12) Patent Application Publication
(10) Pub. No.: US 2005/0147657 A1
(43) Pub. Date: Jul. 7, 2005

(54) White silver-containing wound care device

(75) Inventors: T. Andrew Canada, Campobello, SC (US); Raymond C. Sturm, Spartanburg, SC (US); K. Mark Wiencek, Inman, SC (US); Jason L. Kreider, Boiling Springs, SC (US); Robert L. Schuette, Boiling Springs, SC (US)

Correspondence Address: Charlotte C. Wilson;Legal Department, M-495, 920 Milliken Road, PO Box 1926, Spartanburg, SC, 29304, US

(73) Assignee: Milliken & Company

(21) Appl. No.: 11/068,639

(22) Filed: Feb. 28, 2005

Related U.S. Application Data
(63) Continuation-in-part of application No. 10/640,837, filed on Aug. 14, 2003.
(63) Continuation-in-part of application No. 10/640,918, filed on Aug. 14, 2003.
(63) Continuation-in-part of application No. 10/640,919, filed on Aug. 14, 2003.

(51) Int. Cl. A61L 15/00; A61K 33/38; A61K 33/42

(52) U.S. Cl. 424/445; 424/618; 424/603; 442/123

(57) ABSTRACT

White wound care devices having a topically applied silver-based antimicrobial finish are provided. The finish consists essentially of at least one silver ion releasing compound and at least one binder compound. The finish may be applied to a target substrate, such as a fiber, fabric, or alginate to provide a single layer antimicrobial wound care device, in which the color of the original substrate is substantially maintained after application of the antimicrobial finish. Alternatively, the silver-containing substrate may be combined with one or more additional layers to provide a composite antimicrobial wound care device.

### CROSS-REFERENCE TO RELATED APPLICATIONS

This application is a continuation-in-part of U.S. patent application Ser. No. 10/640,837, entitled “Topical Silver-Based Antimicrobial Composition for Wound Care Devices,” U.S. patent application Ser. No. 10/640,918, entitled “Silver-Containing Wound Care Device,” and U.S. patent application Ser. No. 10/640,919, entitled “Method for Producing a Silver-Containing Wound Care Device,” each of which was filed on Aug. 14, 2003.

### TECHNICAL FIELD

This disclosure relates to wound care devices having a topically applied silver-based antimicrobial finish. More specifically, this disclosure relates to topical antimicrobial finishes with silver ion-releasing mechanisms and to articles having these antimicrobial finishes. The application of the present finish to a substrate results in an antimicrobial product that substantially retains its initial color. This highly desirable feature contrasts sharply with products that are commercially available today and that may be described in the prior art, which are silver-based antimicrobial articles that typically appear as dark colored substrates.

The present finish may be applied to a target substrate to provide a single layer antimicrobial wound care device. Alternatively, a silver-containing layer, as will be described herein, may be combined with one or more additional layers to provide a composite antimicrobial wound care device.

In one potentially preferred embodiment, a silver-based antimicrobial finish is topically applied to a fabric comprised of fibers. Such fibrous substrates provide sufficient surface area onto which the silver ion-releasing antimicrobial may adhere, thus making available an amount of surface-available silver on the wound care device that is sufficient for promotion of wound healing.

In whatever form (that is, single layer or composite) and being made from whatever materials (natural or synthetic), the present wound care device substrate is unique in that it substantially retains its original color through processing, irradiation, and storage, compared to similar articles produced from other silver-based compounds present at the levels required for effective wound treatment. Further, the ability to create essentially white-colored, silver-based antimicrobial textiles affords the opportunity to provide silver-based antimicrobial substrates in a wide variety of light colors previously unavailable.

### BACKGROUND

Silver-containing antimicrobials have been incorporated into wound care devices and are rapidly gaining acceptance in the medical industry as a safe and effective means of controlling microbial growth in the wound bed, often resulting in improved healing. It is known that placing surface-available silver in contact with a wound allows the silver to enter the wound and become absorbed by undesirable bacteria and fungi that grow and prosper in the warm, moist environment of the wound site. Once absorbed, the silver ions kill microbes, resulting in treatment of infected wounds or the prevention of infection in at-risk wounds.

For example, U.S. Pat. No. 3,930,000 discloses the use of a silver zinc allantoinate cream for killing bacteria and fungi associated with burn wounds. Another example is silver sulfadiazine (sold under the tradename SILVADINE®), which has been shown to be effective when tested in vitro against 50 strains of MRSA.

It is also known that silver ion-releasing compounds selected from the group consisting of silver ion exchange materials (e.g. zirconium phosphates and zeolites), silver particles (e.g. silver metal, nanosilver, colloidal silver), silver salts (e.g. AgCl, Ag₂CO₃), silver glass, and mixtures thereof, are generally susceptible to discoloration and have a tendency to alter the color of the substrate in which they are incorporated. More specifically, excess silver ions can combine with available anions to form colored, precipitated salts. Many of these silver salts darken upon exposure to light as a result of the photo-reduction of silver ion to silver metal. When such compounds are incorporated into prior wound care devices at levels required to deliver effective performance, the color of the substrate is darkened as a result of the presence of the silver compounds.

This dark color of the substrate is especially problematic in the medical industry, and specifically in wound care devices, where examination of the wound site (as well as the bandage or dressing covering the wound) can provide important indicators of the effectiveness of the treatment administered to a particular wound. As such, evidence of color on the wound care device may indicate infection at a wound site (e.g., purulent green discharge being indicative of infection with a Pseudomonas species of bacteria), uncontrolled bleeding (e.g., red discharge), or debrided eschar (e.g., brown slough), with such discoloration being more readily apparent in a white, or similarly light-colored, wound care device.

However, if the device has a dark color as manufactured due to a high loading of silver antimicrobial contained within or on the wound care device itself, the relevant color (e.g., from blood or infected exudates) during use may be more difficult for the caregiver to assess. Thus, it is important to those in the medical industry that the wound care device itself does not become discolored merely because silver ions are undergoing photo-reduction, as such discoloration could lead to confusion as to the effectiveness of the treatment being administered to the wound. Accordingly, a stable silver-containing antimicrobial finish on a wound care device, which substantially maintains the original color of the device, is most desirable.

There have been various attempts by others to create silver ion-releasing wound care devices. In many wound care devices, the silver antimicrobial is present throughout the entire cross section of the device. For example, silver antimicrobials have been adapted for incorporation within melt-spun synthetic fibers in order to provide certain fabrics that selectively and inherently exhibit antimicrobial characteristics. Commercial examples include DAK's antimicrobial polyester fiber under the tradename STERIPURE® and Unifi's antimicrobial nylon fiber under the tradename A.M.Y. In another example, silver antimicrobials have been adapted for incorporation within bi-component, core/sheath fibers as taught within U.S. Pat. Nos. 6,723,428 and 6,841,244.

However, the melt-spun fibers described above are expensive to produce due to the large amount of silver-based compound required to provide sufficient antimicrobial activity, especially in light of the relative lack of migration of the compound from within the fiber itself to its surface. As such, when these silver-containing fibers are combined to form a wound care device, the silver located on the interior of the fiber may never reach the wound site during the useful life of the device to provide any advantage to the healing process. Thus, this approach results in an inefficient and expensive use of silver in wound care devices, and it is even likely that the amount of silver released from the fibers is inadequate for promoting the healing process.

Others have attempted to provide composite, multi-layered wound care devices. An example of this approach is marketed by Smith and Nephew under the tradename ACTICOAT™. This wound care device is comprised of three layers—a layer of polyethylene film, a middle layer of rayon/polyester blend nonwoven fabric, and a second layer of film. Nano-crystalline silver particles are deposited onto the film layers to provide an antimicrobial wound care device. However, this technology generally fails to impart desirable release of silver from the device, while the device itself exhibits a metallic blue coloration. This product has the potential to initially release large amounts of silver from the wound care device, often in the form of silver flakes, which may enter the wound bed and may lead to irritation of the wound.

Another product available to consumers, provided by Johnson & Johnson under the trademark ACTISORB®, is a highly porous, silver-impregnated charcoal cloth, sandwiched between two nylon nonwoven layers. This product generally provides very low release of silver, and the device itself is black initially due to the presence of a silver charcoal active ingredient.

Yet another example is manufactured by Argentum under the trademark SILVERLON®. Silver, via a solution of silver nitrate, is reduced and deposited on sensitized polymeric fibers (typically nylon). The silver-laden polyamide is then attached to a subsequent fiber layer. Because of the nature of this technology, it is difficult to control the amount of silver deposited on the substrate, causing this product to show dark coloration as well.

In all cases where the wound care device is colored (e.g., metallic blue, brown, gray, black) by the addition of silver to the device, a situation exists in which medical personnel and/or the users thereof will have greater difficulty in caring for wound sites and monitoring the healing process. To this point, attempts to create a fiber-based wound care device having both effective antimicrobial properties and its original color (preferably white) have been unsuccessful.

Because treatments in which the silver antimicrobial is incorporated into the fiber have been found ineffective, another approach was necessary. A topical finish for textile substrates, such as a fabric, is desirable because it permits treatment of a fabric's individual fibers before or after weaving, knitting, and the like, in order to provide greater functionality to the target yarn and enhanced likelihood of effectiveness in a wound care device. Such a finish should be capable of releasing a desired amount of silver to the wound from a substrate whose color has not been substantially altered by the addition of a silver antimicrobial. Furthermore, it is desirable in the case of metallic silver that a metallized treatment be electrically non-conductive on the target fabric, fiber, or yarn.

Methods of topically applying a silver-based antimicrobial finish to textile substrates are described in commonly assigned U.S. Pat. Nos. 6,584,668 and 6,821,936 and in commonly assigned U.S. patent application Ser. Nos. 09/586,081; 09/589,179; 10/307,027, and 10/306,968. All of these patents and patent applications are hereby incorporated by reference. Details of many of these processes will be discussed below.

The present disclosure addresses and overcomes the problems described above. Whereas, historically, a silver antimicrobial has been incorporated into a melt or polymer matrix prior to the formation of a fiber or substrate, or silver metal is deposited on the surface to create a dark-colored antimicrobial substrate useful for wound care devices, the present disclosure describes a method for achieving an effective wound care device having a silver-based antimicrobial finish, which is topically applied to a target substrate without substantially altering the color of the device. The resultant wound care device provides desired release of silver to the wound site and, because of its unchanged color, offers benefits in terms of wound monitoring and manufacturing flexibility.

The present wound care device optionally includes additional layers that may assist in boosting absorption capacity, such as, for example, one or more layers of cotton gauze, foam, alginate, carboxymethyl cellulose, and the like. These additional layers may or may not contain an antimicrobial agent.

For these reasons and others that will be described herein, the present effective silver wound care device represents a useful advance over the prior art.

### BRIEF DESCRIPTION OF THE DRAWINGS

FIG. 1 is a line graph depicting the reflectance of Examples 2A, 3A, 4A, 5B, and Comparative Examples A-E.

### DETAILED DESCRIPTION

### Wound Care Substrate

Suitable substrates for receiving a topically applied silver-based antimicrobial finish include, without limitation, fibers, fabrics, and alginates. The fabric may be formed from fibers such as synthetic fibers, natural fibers, or combinations thereof. Synthetic fibers include, for example, polyester, acrylic, polyamide, polyolefin, polyaramid, polyurethane, regenerated cellulose (i.e., rayon), and blends thereof.

The term “polyamide” is intended to describe any long-chain polymer having recurring amide groups (—NH—CO—) as an integral part of the polymer chain. Examples of polyamides include nylon 6; nylon 6, 6; nylon 1, 1; and nylon 6, 10.

The term “polyester” is intended to describe any long-chain polymer having recurring ester groups (—C(O)—O—). Examples of polyesters include aromatic polyesters, such as polyethylene terephthalate (PET), polybutylene terephthalate (PBT), polytrimethylene terephthalate (PTT), and polytriphenylene terephthalate, and aliphatic polyesters, such as polylactic acid (PLA).

“Polyolefin” includes, for example, polypropylene, polyethylene, and combinations thereof. “Polyaramid” includes, for example, poly-p-phenyleneteraphthalamid (i.e., Kevlar®), poly-m-phenyleneteraphthalamid (i.e., Nomex®), and combinations thereof. Natural fibers include, for example, wool, cotton, flax, and blends thereof.

The fabric may be formed from fibers or yarns of any size, including microdenier fibers and yarns (fibers or yarns having less than one denier per filament). The fibers or yarns may have deniers that range from less than about 1 denier per filament to about 2000 denier per filament or more preferably, from less than about 1 denier per filament to about 500 denier per filament, or even more preferably, from less than about 1 denier per filament to about 300 denier per filament.

Furthermore, the fabric may be partially or wholly comprised of multi-component or bi-component fibers or yarns, which may be splittable, or which have been partially or fully split, along their length by chemical or mechanical action. The fabric may be comprised of fibers such as staple fiber, filament fiber, spun fiber, or combinations thereof.

The fabric may be of any variety, including but not limited to, woven fabric, knitted fabric, nonwoven fabric, or combinations thereof. The unique and interesting achievement realized through the present topical application is that the wound care device substantially maintains its original color, despite the presence of effective amounts of a silver-based antimicrobial agent.

The elimination of color normally associated with the inclusion of silver-based antimicrobials is highly beneficial and desirable. The wound care devices (preferably, white-colored), as will be described herein, allow users thereof and their health care providers to monitor the exudates from the wound. Further, the present wound care devices exhibit long-term color stability (that is, their color does not change significantly over time while in production, transit, or storage). Finally, because the present wound care device is not discolored by the addition of the silver-based antimicrobial agent, a variety of substrate colors may be utilized or the finished wound care devices may be dyed or colored to any desired shade or hue with any type of colorant, such as, for example, pigments, dyes, tints, and the like.

For instance, the fabric used for the present wound care device may optionally be colored by a variety of dyeing techniques, such as high temperature jet dyeing with disperse dyes, vat dyeing, thermosol dyeing, pad dyeing, transfer printing, screen printing, or any other technique that is common in the art for comparable textile products. If yarns or fibers are treated by the process of the current invention, they may be dyed by suitable methods prior to fabric formation, such as, for instance, by package dyeing or solution dyeing, or after fabric formation as described above, or they may be left undyed.

Other additives may be present on and/or within the target fabric or yarn, including antistatic agents, optical brightening compounds, opacifiers (such as titanium dioxide), nucleating agents, antioxidants, UV stabilizers, fillers, permanent press finishes, softeners, lubricants, curing accelerators, adhesives, and the like. The present fabrics may also be coated or printed or otherwise aesthetically modified in addition to being treated with the present antimicrobial compositions.

Alginates from commercial sources are an alternative substrate, which may be used in place of fabrics. A typical process of manufacturing alginates involves crushing and washing of the raw material (i.e., seaweed) and dissolution of the extracted sodium alginate in water. A viscous solution is obtained that is extruded into a calcium chloride bath. Here, the sodium ions are replaced by calcium ions, and an insoluble calcium alginate is precipitated. Rinsing and dehydration then leads to the production of a fiber. Fibers may be formed from alginate by extruding or spinning the alginate from an aqueous solution. The fibers are then typically laid down in a web mat that can be incorporated into a wound care device.

An alginate web containing the calcium alginate fibers is placed on the wound in a dry state and begins to absorb the exudates. At this time, reverse ion exchange takes place, in which the calcium ions that are present in the alginate are gradually exchanged for sodium ions that are present in the blood and wound exudates. The fibers absorb large amounts of secretions, start to swell, and, in the presence of sodium ions, turn into a moist gel that fills and securely covers the wound.

In one embodiment of the invention, a commercially available nonwoven fabric is used to form the wound care device. Nonwovens are known in the textile industry as an alternative to traditional woven or knit fabrics. To create a nonwoven fabric, a filament web must be created and then consolidated. In one method, staple fibers are formed into a web through the carding process, which can occur in either wet or dry conditions. Alternatively, continuous filaments, which are formed by extrusion, may be used in the formation of the web. The web is then consolidated, and/or bonded, by means of needle-punching, thermal bonding, chemical bonding, or hydroentangling. A second consolidation method may also be employed such as thermal bonding.

One preferred substrate for use in the wound care device of the present disclosure is a nonwoven fabric formed of continuous splittable filaments that are extruded as a web and then consolidated. This nonwoven fabric is described in U.S. Pat. Nos. 5,899,785 and 5,970,583, both assigned to Firma Carl Freudenberg of Weinheim, Germany. Preferably, the nonwoven web is consolidated through hydroentanglement, and, more preferably, through hydroentanglement followed by thermal, or point, bonding. The continuous composite filaments are obtained by means of a controlled spinning process, and the hydroentanglement process mechanically splits at least some, if not most, of the composite filaments into their elementary components. This structure of split fibers provides a greater surface area onto which the present silver-based antimicrobial compound may be applied and, therefore, greater amounts of surface-available silver that may contact the wound.

While a potentially preferred nonwoven fabric has been described, it is believed that any fiber or fabric that has been treated with the silver-based antimicrobial chemistry described herein is suitable for use within the present wound care device, as well as any of the above-mentioned substrate materials.

### Antimicrobial and Other Agents

The particular treatment used herein comprises at least one silver ion-releasing compound selected from the group consisting of silver ion exchange materials (e.g. zirconium phosphates and zeolites), silver particles (e.g. silver metal, nanosilver, colloidal silver), silver salts (e.g. AgCl, Ag₂CO₃), silver glass, and mixtures thereof. One preferred silver ion-containing compound is an antimicrobial silver sodium hydrogen zirconium phosphate available from Milliken & Company of Spartanburg, S.C., sold under the tradename “ALPHASAN®”. Other potentially preferred silver-containing antimicrobials suitable for use herein—including silver zeolites, such as a silver ion-loaded zeolite available from Sinanen Co., Ltd. of Tokyo, Japan under the tradename “ZEOMIC”, and silver glass, such as those available from Ishizuka Glass Co., Ltd. of Japan under the tradename “IONPURE®”—may be utilized either in addition to, or as a substitute for, the preferred species listed above. Other silver ion-containing materials may also be used. Various combinations of these silver-containing materials may be made if adjustments to the silver release rate over time are desired.

Generally, the silver-based compound is added in an amount from about 0.01% to about 60% by total weight of the particular finish composition; more preferably, from about 0.05% to about 40%; and most preferably, from about 0.1% to about 30%. The antimicrobial finish itself, including any desired binders, wetting agents, odor absorbing agents, leveling agents, adherents, thickeners, and the like, is added to the substrate in an amount of at least about 0.01% of the total device weight.

A binder material has been found useful in preventing the antimicrobial from flaking onto the wound. Preferably, this component is a polyurethane-based binding agent, although a wide variety of cationic, anionic, and non-ionic binders may also be used, either alone or in combination. In essence, such binders provide durability by adhering the antimicrobial to the target substrate, such as fibers or fabrics, without negatively affecting the release of silver ions to the wound.

Total add-on levels of silver to the target substrate may be 20 ppm or higher. More preferably, total add-on levels of silver may be 200 ppm or higher. Although an upper boundary limit of silver add-on levels to the target substrate has not been determined, consideration of the manufacturing economics and the potential to irritate a sensitive wound site suggests avoiding excessive silver levels.

### Application of Antimicrobial and Other Agents to Substrate

Preferably, silver ion-containing compounds (such as ALPHASAN®, ZEOMIC®, or IONPURE®) are admixed in an aqueous dispersion with a binder to form a bath into which the target substrate is immersed. Other similar types of compounds that provide silver ions may also be utilized.

When specific polyurethane-based binder materials are utilized, the antimicrobial characteristics of the treated substrate are effective with regard to the amount of surface available silver that is released to kill bacteria, without altering the color of the treated substrate (that is, while substantially maintaining its original appearance). While it currently appears that the use of polyurethane-based binder resins are preferred due to their allowance of silver release and bio-neutral properties, in practice essentially any effective cationic, anionic, or non-ionic binder resin that is not toxic to the wound may be used.

An acceptable method of providing a durable antimicrobial silver-treated fabric surface is the application of a silver ion-containing compound and polyurethane-based binder resin from a bath mixture. This mixture of antimicrobial compound and binder resin may be applied through any technique as is known in the art, including spraying, dipping, padding, foaming, printing, and the like.

The following examples further illustrate the present antimicrobial device but are not to be construed as limiting the invention as defined in the claims appended hereto. All parts and percents given in these examples are by weight unless otherwise indicated.

### Sample Creation and Evaluation

A. Substrate Descriptions

The fiber used in Example 1 was a 70 denier 34 filament Dacron® polyester fiber.

The fabric used in Examples 2A-2E and Example 2Control was a nonwoven fabric comprised of natural and synthetic fibers. The fabric weight is approximately 68 g/m². The fabric is manufactured and sold by Ahlstrom.

The fabric used in Examples 3A-3B and Example 3 Control was a point-bonded nonwoven fabric, having a fabric weight of 130 g/m² and sold under the tradename EVOLON® by Firma Carl Freudenberg of Weinheim, Germany. Polyester fiber comprised about 65% of the fabric, and nylon 6,6 fiber comprised about 35% of the fabric. The fabric was not dyed.

The fabric used in Examples 4A-4D and Example 4 Control was a nonwoven fabric made of 100% polyester having a weight of approximately 40 g/m². The fabric is sold under the tradename CELFIL® by Polimeros y Derivados. As purchased (prior to addition of an antimicrobial composition), the fabric contained optical brightening agents to enhance the fabric's brightness.

The fabric used in Examples 5A-5B, Example 5 Control, Example 6, and Examples 7A-7F was a multi-polymer fabric sold by Milliken & Company. The circular knit fabric was comprised of 66% nylon-6, 19% polyester, and 15% spandex, and was knitted in such as manner as to give a distinct nylon side and a distinct polyester side.

B. Antimicrobial Coating Formulations

Various dispersions of an antimicrobial finish include combinations of the following components:
ALPHASAN® RC2000 silver-based ion exchange compound, available from Milliken & Company of Spartanburg, S.C.
Aqueous dispersion of nanosilver particles having an average particle size of between 20 nm and 80 nm, available from CIMA Nanotech of St. Paul, Minn.
Witcobond® polyurethane binders available from Crompton of Middlebury, Conn.
Lubril QCJ, a hydrophilic polymer dispersion available from Roebuck Operations of Spartanburg, S.C.
Freecat MX, an aqueous white liquid consisting of a buffered magnesium salt available from Noveon Chemicals of Cleveland, Ohio

### EXAMPLE 1

A 70-denier, 34 filament Dacron® polyester fiber was used for Example 1. A solution was prepared according to the formulation in TABLE 1 and was applied to the polyester fiber using an Atlab finish applicator manufactured by Atlas Industries. Prior to testing, 12 strands of this fiber were hand twisted into a yarn about 5 cm in length. The resulting fiber had a 7.5% wt/wt content of AlphaSan® RC2000.

TABLE 1

| Component | Amount (grams) |
|---|---|
| Water | 37 |
| Witcobond 293 (polyurethane binder) | 1.5 |
| AlphaSan ® RC 2000 (antimicrobial agent, 10% Ag) | 1.5 |

### EXAMPLES 2A-2E

The AHLSTROM® nonwoven fabric described above was coated using the formulations shown below in TABLE 2. Examples 2A through 2E were prepared using the following steps:
(a) The coating solution was prepared at room temperature via stirring the components listed below in a container for approximately one hour; and
(b) the fabric was dipped in a bath, squeezed via nip rollers, and dried in an oven at around 350° F. for between two and three minutes.

A Control sample (Example 2 Control) was also prepared in a water-only solution, which was exposed to the same process conditions as Examples 2A-2E.

TABLE 2
Formulations for Examples 2A-2E

| | Components (grams) | | | | | % Active |
|---|---|---|---|---|---|---|
| | | AlphaSan ® | Witcobond | Lubril | | Alphasan ® |
| Sample ID | Water | RC 2000 | 293 | QCX | Freecat MX | (wt/.wt %) |
| Example 2A | 365.3 | 100.0 | 15.0 | 9.8 | 10.0 | 20.7% |
| Example 2B | 409.5 | 51.2 | 19.1 | 10.0 | 10.2 | 8.2% |
| Example 2C | 417.9 | 25.0 | 37.3 | 9.8 | 10.0 | 4.2% |
| Example 2D | 398.3 | 5.1 | 76.4 | 10.0 | 10.0 | 1.1% |
| Example 2E | 477.7 | 5.0 | 7.5 | 9.78 | 0.08 | 1.0% |

NOTE:
The weight/weight % is calculated by dividing the weight of antimicrobial agent as determined by analytical procedure by the weight of the dry coated fabric.

### EXAMPLES 3A-3B

The EVOLON® nonwoven fabric described above was coated using the formulations shown below in TABLE 3. These Examples were prepared via the same process used to create the finished fabrics of Example 2. A Control sample (Example 3 Control) was also prepared in a water-only solution, which was exposed to the same process conditions as Examples 3A-3B.

TABLE 3
Formulations for Examples 3A-3B

| | Components (grams) | | | | | % Active |
|---|---|---|---|---|---|---|
| | | AlphaSan ® | Witcobond | Lubril | | Alphasan ® |
| Sample ID | Water | RC 2000 | 293 | QCX | Freecat MX | (wt./wt. %) |
| Example 3A | 961.1 | 444.8 | 331.7 | 173.9 | 88.9 | 18.2% |
| Example 3B | 1740.3 | 111.2 | 82.9 | 43.5 | 22.2 | 2.2% |

NOTE:
The weight/weight % is calculated by dividing the weight of antimicrobial agent by the weight of the coated fabric.

### EXAMPLES 4A-4D

The CELFIL® nonwoven fabric described above was coated using the formulations shown below in TABLE 4. These Examples were prepared via the same process used to create the finished fabrics of Example 2. A Control sample (Example 4 Control) was also prepared in a water-only solution, which was exposed to the same process conditions. The fabric used to create Examples 4A-4D and Example 4 Control included an optical brightener to enhance its brightness.

TABLE 4
Formulations for Examples 4A-4D

| | Components (grams) | | | | | % Active |
|---|---|---|---|---|---|---|
| | | AlphaSan ® | Witcobond | Lubril | | Alphasan ® |
| Sample ID | Water | RC 2000 | 293 | QCX | Freecat MX | (wt./wt %) |
| Example 4A | 1707.1 | 175.5 | 65.5 | 34.3 | 17.5 | 16.0% |
| Example 4B | 456.4 | 25.0 | 18.7 | 0 | 0 | 9.9% |
| Example 4C | 288.4 | 25.0 | 186.6 | 0 | 0 | 5.0% |
| Example 4D | 457.7 | 5.0 | 37.3 | 0 | 0 | 1.2% |

NOTE:
The weight/weight % is calculated by dividing the weight of antimicrobial agent as determined by analytical procedure by the weight of the dry coated fabric.

### EXAMPLES 5A and 5B

The multi-polymer fabric described above was coated using the formulations shown below in TABLE 5. These Examples were prepared via the same process used to create the finished fabrics of Example 2. In Example 5A, an optical brightener was included as part of the material (before application of the antimicrobial composition). Example 5B did not include an optical brightener.

Two Control samples (Example 5A Control, which contained an optical brightener, and Example 5B Control, which did not contain an optical brightener) were also prepared in a water-only solution, which was exposed to the same process conditions as Examples 5A and 5B.

Measurements of the Example fabrics and the Control fabrics were made from both the nylon side of the material and the polyester side of the material.

TABLE 5
Formulations for Examples 5A-5B

| | Components (grams) | | | | |
|---|---|---|---|---|---|
| | | | | Optical | % Active |
| | | AlphaSan ® | Witcobond | Bright- | Alphasan ® |
| Sample ID | Water | RC 2000 | 293 | ener | (wt./wt %) |
| Example | 3083.4 | 705.9 | 210.7 | Yes | 14.4 |
| 5A | | | | | |
| Example | 2083.4 | 705.9 | 210.7 | No | 20.1 |
| 5B | | | | | |

The weight/weight % is calculated by dividing the weight of antimicrobial agent as determined by analytical procedure by the weight of the dry coated fabric.

### EXAMPLE 6

### Nanoparticle Silver

The fabric used in Example 6 was the multi-polymeric fabric of Example 5. This Example was prepared using the same process used to create the fabrics of Example 2. The formulation for this Example is shown in TABLE 6.

TABLE 6
Nanoparticle Silver Formulation

| Component | Amount (grams) |
|---|---|
| Water | 191 |
| Witcobond 290H (polyurethane binder) | 5.3 |
| Cima NanoTech product no. AB120-1 (antimicrobial | 3.5 |
| agent) | |

### EXAMPLES 7A-7F

The fabric used in Example 7 was the multi-polymeric fabric used in Example 5. They were dyed with one of the following pastel dye colors and concentrations.

| | |
|---|---|
| 7A: | Pastel blue color; full dye concentration |
| 7B: | Pastel blue color; half dye concentration |
| 7C: | Pastel green color; full dye concentration |
| 7D: | Pastel green color; half dye concentration |
| 7E: | Pastel purple color; full dye concentration |
| 7F: | Pastel purple color; half dye concentration |

Examples 7A-7F were coated with the same antimicrobial finish (formulation shown in TABLE 7). Control samples, corresponding to each of the dyed samples and having the same dye color and amount, were also created and subjected to the same processing conditions.

TABLE 7
Formulation used for Examples 7A-7F

| Component | Amount (grams) |
|---|---|
| Water | 1388.1 |
| Witcobond 293 (polyurethane binder) | 141.2 |
| AlphaSan ® RC 2000 (antimicrobial agent, 10% Ag) | 471.2 |

C. Comparative Sample Descriptions

Several commercially available silver-containing wound care devices were also purchased for evaluation. These textile-based wound care devices are notated as Comparative Examples A-E below and include a wide variety of wound dressing combinations.

### COMPARATIVE EXAMPLE A

“Actisorb 220”, a multi-component nonwoven wound care device comprised of a highly porous, silver impregnated charcoal cloth sandwiched between two nylon nonwoven layers containing 220 mg of silver; available from Johnson & Johnson of Somerville, N.J.

### COMPARATIVE EXAMPLE B

“Acticoat 5”, a three layered wound care device having a rayon/polyester blend layer of nonwoven fabric sandwiched between two layers of nanocrystalline silver-coated polyethylene film; available from Smith and Nephew of Largo, Fla.

### COMPARATIVE EXAMPLE C

“Acticoat 7”, a five-layered wound care device similar to “Acticoat 5” that has additional layers of fabric and film; also available from Smith and Nephew of Largo, Fla.

### COMPARATIVE EXAMPLE D

“Silverlon”, a silver-plated nylon fabric; available from Argentum Medical, LLC of Lakemont, Ga.

### COMPARATIVE EXAMPLE E

“Aquacel Ag”, a silver-impregnated sodium carboxymethyl cellulose hydrofiber having 1.2% silver; available from Convatec, a Bristol-Myers-Squibb Company of England.

D. Example Testing and Evaluation

Each of the above examples were tested for a variety of characteristics as will be described below. Further, commercially available products (referred to as Comparative Examples A-E and described above) were also tested for comparison with the present antimicrobial wound care substrates. The testing procedures will be described in detail as follows. However, a listing of the tests used is found below.
Test 1. Zone of Inhibition Testing (Kirby-Bauer Agar Diffusion Assay)
Test 2. Quantitative Log Reduction (Modified AATCC Method 100)
Test 3. Wavelength/Reflectance Evaluation
Test 4. Whiteness/Yellowness Evaluation
Test 5. Color Evaluation: Lightness/Darkness, Yellow/Blue, Red/Green
Test 6. Comparison of Magnitude of Color Difference between Sample and Reference Tile
Test 7. Color Stability Testing

### TEST 1: Zone of Inhibition Test (Kirby-Bauer Agar Diffusion Assay)

Examples were tested against one or more of Staphylococcus aureus ATCC #6538, Pseudomonas aeruginosa ATCC #12055, using a standard zone of inhibition test based on the Kirby-Bauer Agar-Diffusion Assay. The procedure is described in the report “Antibiotic Susceptibility Testing by a Standardized Single Disc Method” written by A. W. Bauer, W. M. Kirby, and M. Truck and published in the American Journal of Clinical Pathology 1966; Volume 45, page 493.

The Zone Of Inhibition (ZOI) test based on the Kirby-Bauer Agar Diffusion Assay provides both a qualitative (level of growth underneath sample) and quantitative (size of zone in millimeters) assessment of the performance of an antimicrobial agent incorporated into a wound dressing. The level of growth underneath the sample can be rated from confluent (“no activity”) to spotty or isolated (“bacteriostatic”) to nil (“bactericidal”). If reduced growth is observed underneath the sample for a particular microorganism when compared to an untreated control dressing, that microorganism is considered sensitive and the antimicrobial agent is effective (i.e., is bacteriostatic). The magnitude of the zone of inhibition, if one is observed, is a measure of both the inherent efficacy of the agent and the diffusion of the agent through the nutrient agar matrix. This zone of inhibition assay can be used to measure the efficacy of the dressings in a simulated clinical application by subjecting the dressings to multiple insults of a high level of bacteria over a period of seven days. For purposes of discussion herein, a substrate is considered to have “effective antimicrobial” properties if it produces a ZOI against bacteria of at least 1 mm after two successive exposures.

Petri dishes containing Diagnostic Sensitivity Test (DST) agar were inoculated via spreading with 0.5 mL of a diluted overnight culture of approximately 5E+05 cells/mL of the test organism into 100 mM Na/K phosphate buffer. An approximately 1 inch by 1 inch piece of the Example fabric was then placed at the center of each agar plate. The agar plates were incubated for 24 hours at 37° C., after which the level of efficacy was determined. To simulate repeated exposure of the dressings to microbes, the dressings were removed from the incubated agar plate, placed onto freshly inoculated agar plates, and incubated another 24 hours. This process was repeated for up to 7 days.

In some cases, an untreated (i.e., without antimicrobial finish) fabric was also tested according to this method. The Control sample was generally a Medisponge® polyurethane foam available from Lendell, an antimicrobial-free substrate.

Zone of Inhibition testing was conducted to determine the antimicrobial activity of the Examples against Staphylococcus aureus. The results, which are shown in TABLE 8, represent an average of four measurements for each sample (one from each of the four sides of the square sample).

TABLE 8
Antimicrobial Activity of Examples 1-5 and Comparative Examples A-E
Against Staphylococcus aureus as Determined By Zone of Inhibition Method (DST)

| | Average | Average | Average | Average | Average | Average | Average |
|---|---|---|---|---|---|---|---|
| | Day 1 | Day 2 | Day 3 | Day 4 | Day 5 | Day 6 | Day 7 |
| | Zone | Zone | Zone | Zone | Zone | Zone | Zone |
| Sample | (mm) | (mm) | (mm) | (mm) | (mm) | (mm) | (mm) |
| Example 1 | 3.0 | n/d | n/d | n/d | n/d | n/d | n/d |
| Example 2A | 9 | 5 | 7 | 7 | 5 | 5 | 4 |
| Example 2B | 10 | 6 | 6 | 5 | 2 | 0 | 0 |
| Example 2C | 9 | n/d | 5 | 3 | 1 | 0 | 0 |
| Example 2D | 7 | 1 | 0 | 0 | 0 | 0 | 0 |
| Example 2 - 3 Control | 0 | 0 | 0 | 0 | 0 | 0 | 0 |
| Example 3A | 4 | 3 | 3 | 3 | 3 | 2 | 4 |
| Example 3B | 4 | 3 | 3 | 3 | 2 | 2 | 5 |
| Example 3 - Control | 0 | 0 | 0 | 0 | 0 | 0 | 0 |
| Example 4A | 7 | 5 | 4 | 4 | n/d | n/d | n/d |
| Example 4B | 3 | 3 | 1 | 0 | n/d | n/d | n/d |
| Example 4C | 0 | 0 | 0 | 0 | n/d | n/d | n/d |
| Example 4D | 0 | 0 | 0 | 0 | n/d | n/d | n/d |
| Example 4 - 3 Control | 0 | 0 | 0 | 0 | 0 | 0 | 0 |
| Example 5B | 5 | 4 | 4 | 4 | 4 | 3 | 4 |
| Example 5 - Control | 0 | 0 | 0 | 0 | 0 | 0 | 0 |
| Comparative Ex. A | 0 | 0 | 0 | 0 | n/d | n/d | n/d |
| Comparative Ex. B | 4 | 4 | 4 | 5 | 5 | 4 | 4 |
| Comparative Ex. C | 6 | 8 | 7 | 7 | 7 | 5 | 9 |
| Comparative Ex. D | 5 | 6 | 5 | 5 | n/d | n/d | n/d |
| Comparative Ex. E | 8 | 5 | 6 | 6 | 7 | 4 | 3 |

NOTE:
“n/d” indicates that the value was not determined.

The results demonstrate that the present Examples 1-5, all of which contained ALPHASAN® RC 2000, were antimicrobially active against Staphylococcus aureus. The control samples, which contained no antimicrobial agent, did not demonstrate antimicrobial activity against any of the bacteria.

### TEST 2: Quantitative Log Reduction (Modified AATCC Method 100)

Examples 2 and 3 and Comparative Examples C and E were tested for antimicrobial performance. Efficacy against bacteria was assessed using a modified version of AATCC Method 100-1999. Portions (approximately 0.5 g) of each fabric were placed in glass vials and exposed to two types of bacteria—Staphylococcus aureus ATCC #6538 (0.5 mL of 1.31E+06 cells/mL) or Pseudomonas aeruginosa ATCC #12055 (0.5 ml of 2.63E+05 cells/mL)—each of which was suspended in a solution of 2% Bovine Serum Albumin in 0.85% NaCl for 18-22 hours at 37° C. After incubation, the samples were washed to remove attached cells. The number of viable cells in the wash solution was quantified using a microtiter plate-based “Most Probable Number” assay. The results are shown in TABLE 9. Negative values, as shown for the Control, indicate bacterial growth.

TABLE 9
Efficacy of Antimicrobial Wound Care Devices
As Determined By Log Reduction Kill Values

| Sample ID | S. aureus | P. aeruginosa |
|---|---|---|
| Example 2A | 2.94 | n/d |
| Example 2B | 2.57 | n/d |
| Example 2C | 1.57 | n/d |
| Example 2D | 3.57 | n/d |
| Example 2 - Control | −1.06 | n/d |
| Example 3A | 1.36 | 1.69 |
| Example 3B | 2.02 | 2.21 |
| Example 3 - Control | −0.89 | −2.06 |
| Comparative Example C | 4.34 | 4.31 |
| Comparative Example E | 0.8 | n/d |

n/d = not determined

### Evaluating Color: Present example devices vs. Comparative samples

As has been previously discussed, providing an essentially white-colored, antimicrobial wound care device is desirable for a number of reasons. First, the antimicrobial properties of the device will facilitate the healing of the wound. This benefit has been described in detail herein. Secondly, by providing an essentially white-colored device, the health care provider and/or user are better able to monitor the exudates from the wound for signs of color change that could indicate a problem.

The wound care device of the present invention exhibits an unusual whiteness when compared to competitive silver-containing antimicrobial devices. This is, in part, due to the specific silver ion-releasing mechanism of the present finish. As will be discussed herein, the white color of the present wound care device is substantially consistent despite the add-on level of the silver-containing antimicrobial agent and is maintained over its shelf-life. Examples 2-7, as well as Comparative Examples A-E, were evaluated for color using various measurements, as will be described herein.

### TEST 3: Wavelength/Reflectance Evaluation

Examples 2A-2E, 3A-3B, 4A-4D, 5A-5B, 6, and 7A-7F were evaluated for color by measuring their reflectance across a given span of wavelengths corresponding to the visible spectrum (i.e., 360 nm-750 nm). Control samples (i.e., fabrics containing no antimicrobial) for each of the present Examples were also evaluated. This evaluation was accomplished via use of a GretagMacbeth Color Eye 70001 spectrophotometer with a D-65 light source and a 10° observation angle. The Comparative Examples were also evaluated, using the same equipment. The reflectance data for Examples 2-5 and the Comparative Examples is shown in TABLES 10-14 below. This data was used to calculate Stensby Index for Whiteness and other comparisons below. Note that a higher reflectance percent indicates a lighter/whiter color.

It should also be noted that reflectance data, although not shown, was generated for Examples 6 and 7A-7F and serves as the basis for the other color measurements of those Examples as will follow.

TABLE 10
Reflectance of Examples 2A-2D and Ex. 2 Control

| Wavelength | Example 2A | Example 2B | Example 2C | Example 2D | Ex. 2 Control |
|---|---|---|---|---|---|
| (nm) | (20.7%) | (8.2%) | (4.2%) | (1.1%) | (0%) |
| 360 | 62.066 | 50.841 | 55.029 | 48.954 | 51.407 |
| 370 | 68.091 | 61.149 | 62.708 | 56.198 | 63.494 |
| 380 | 72.152 | 67.144 | 68.933 | 62.251 | 70.499 |
| 390 | 75.398 | 71.047 | 74.110 | 67.510 | 74.569 |
| 400 | 78.111 | 74.093 | 77.822 | 71.604 | 77.448 |
| 410 | 80.473 | 76.755 | 80.397 | 74.683 | 79.681 |
| 420 | 82.628 | 79.207 | 82.360 | 77.209 | 81.532 |
| 430 | 84.419 | 81.338 | 83.862 | 79.293 | 83.010 |
| 440 | 85.974 | 83.181 | 85.157 | 81.112 | 84.295 |
| 450 | 87.339 | 84.760 | 86.263 | 82.703 | 85.451 |
| 460 | 88.563 | 86.118 | 87.235 | 84.135 | 86.465 |
| 470 | 89.669 | 87.329 | 88.118 | 85.428 | 87.385 |
| 480 | 90.627 | 88.389 | 88.855 | 86.598 | 88.172 |
| 490 | 91.365 | 89.216 | 89.421 | 87.526 | 88.785 |
| 500 | 92.090 | 90.015 | 89.987 | 88.436 | 89.406 |
| 510 | 92.582 | 90.586 | 90.391 | 89.096 | 89.840 |
| 520 | 92.962 | 91.029 | 90.698 | 89.610 | 90.192 |
| 530 | 93.273 | 91.407 | 90.960 | 90.053 | 90.515 |
| 540 | 93.563 | 91.781 | 91.242 | 90.459 | 90.821 |
| 550 | 93.742 | 92.034 | 91.406 | 90.752 | 91.010 |
| 560 | 93.909 | 92.252 | 91.571 | 91.029 | 91.193 |
| 570 | 94.030 | 92.424 | 91.694 | 91.254 | 91.326 |
| 580 | 94.155 | 92.584 | 91.824 | 91.462 | 91.472 |
| 590 | 94.270 | 92.733 | 91.968 | 91.648 | 91.632 |
| 600 | 94.349 | 92.848 | 92.082 | 91.833 | 91.768 |
| 610 | 94.338 | 92.869 | 92.100 | 91.903 | 91.812 |
| 620 | 94.439 | 92.991 | 92.222 | 92.065 | 91.938 |
| 630 | 94.482 | 93.082 | 92.304 | 92.184 | 92.029 |
| 640 | 94.585 | 93.212 | 92.435 | 92.361 | 92.175 |
| 650 | 94.608 | 93.258 | 92.482 | 92.442 | 92.234 |
| 660 | 94.685 | 93.370 | 92.590 | 92.587 | 92.362 |
| 670 | 94.693 | 93.390 | 92.622 | 92.650 | 92.421 |
| 680 | 94.699 | 93.436 | 92.676 | 92.712 | 92.475 |
| 690 | 94.672 | 93.425 | 92.684 | 92.733 | 92.466 |
| 700 | 94.617 | 93.405 | 92.649 | 92.736 | 92.465 |
| 710 | 94.595 | 93.395 | 92.660 | 92.731 | 92.452 |
| 720 | 94.548 | 93.378 | 92.636 | 92.746 | 92.440 |
| 730 | 94.668 | 93.492 | 92.772 | 92.866 | 92.552 |
| 740 | 94.762 | 93.605 | 92.859 | 93.002 | 92.681 |
| 750 | 94.908 | 93.826 | 92.966 | 93.274 | 92.913 |

TABLE 11
Reflectance of Examples 3A-3B and Example 3 Control

| Wavelength | Example 3A | Example 3B | Example 3 |
|---|---|---|---|
| (nm) | (18.2%) | (2.2%) | Control (0%) |
| 360 | 36.197 | 44.088 | 46.48 |
| 370 | 50.591 | 60.687 | 64.103 |
| 380 | 64.071 | 72.756 | 76.585 |
| 390 | 74.915 | 79.579 | 83.110 |
| 400 | 81.752 | 83.286 | 86.580 |
| 410 | 85.343 | 85.385 | 88.712 |
| 420 | 87.416 | 86.731 | 90.188 |
| 430 | 88.773 | 87.571 | 91.196 |
| 440 | 89.879 | 88.266 | 92.054 |
| 450 | 90.812 | 88.865 | 92.785 |
| 460 | 91.622 | 89.346 | 93.400 |
| 470 | 92.349 | 89.828 | 93.968 |
| 480 | 92.938 | 90.225 | 94.449 |
| 490 | 93.319 | 90.504 | 94.758 |
| 500 | 93.750 | 90.849 | 95.098 |
| 510 | 94.034 | 91.084 | 95.292 |
| 520 | 94.227 | 91.266 | 95.440 |
| 530 | 94.405 | 91.448 | 95.559 |
| 540 | 94.611 | 91.649 | 95.692 |
| 550 | 94.711 | 91.769 | 95.751 |
| 560 | 94.821 | 91.896 | 95.812 |
| 570 | 94.902 | 92.013 | 95.858 |
| 580 | 95.004 | 92.152 | 95.926 |
| 590 | 95.089 | 92.290 | 95.977 |
| 600 | 95.122 | 92.398 | 96.008 |
| 610 | 95.079 | 92.439 | 95.946 |
| 620 | 95.159 | 92.594 | 96.019 |
| 630 | 95.202 | 92.705 | 96.030 |
| 640 | 95.297 | 92.893 | 96.123 |
| 650 | 95.328 | 92.996 | 96.138 |
| 660 | 95.407 | 93.133 | 96.200 |
| 670 | 95.367 | 93.184 | 96.163 |
| 680 | 95.386 | 93.269 | 96.162 |
| 690 | 95.373 | 93.306 | 96.100 |
| 700 | 95.296 | 93.311 | 95.989 |
| 710 | 95.290 | 93.359 | 95.955 |
| 720 | 95.253 | 93.386 | 95.895 |
| 730 | 95.366 | 93.561 | 96.014 |
| 740 | 95.480 | 93.745 | 96.118 |
| 750 | 95.638 | 93.981 | 96.270 |

TABLE 12
Reflectance of Examples 4A-4D and Example 4 Control

| | Example | Example | Example | Example | Example 4 |
|---|---|---|---|---|---|
| Wavelength | 4A | 4B | 4C | 4D | Control |
| (nm) | (16.0%) | (9.9%) | (5.0%) | (1.2%) | (0%) |
| 360 | 26.958 | 24.228 | 18.745 | 22.122 | 22.65 |
| 370 | 27.573 | 24.349 | 19.639 | 22.244 | 22.639 |
| 380 | 27.624 | 23.949 | 20.667 | 21.909 | 22.143 |
| 390 | 31.171 | 27.224 | 24.934 | 25.104 | 25.104 |
| 400 | 37.445 | 33.583 | 31.988 | 31.377 | 30.986 |
| 410 | 55.503 | 52.738 | 51.249 | 50.262 | 49.482 |
| 420 | 88.135 | 88.755 | 85.541 | 85.464 | 86.578 |
| 430 | 109.019 | 112.592 | 107.557 | 108.292 | 112.049 |
| 440 | 117.182 | 121.542 | 115.378 | 116.723 | 121.699 |
| 450 | 110.478 | 113.787 | 108.641 | 109.135 | 113.782 |
| 460 | 102.865 | 105.017 | 101.231 | 100.680 | 104.566 |
| 470 | 100.722 | 102.352 | 99.020 | 98.164 | 101.770 |
| 480 | 98.470 | 99.602 | 96.763 | 95.621 | 98.900 |
| 490 | 94.931 | 95.490 | 93.347 | 91.809 | 94.557 |
| 500 | 93.863 | 94.154 | 92.342 | 90.712 | 93.110 |
| 510 | 92.692 | 92.754 | 91.226 | 89.585 | 91.643 |
| 520 | 91.088 | 90.927 | 89.723 | 88.054 | 89.733 |
| 530 | 89.835 | 89.511 | 88.534 | 86.897 | 88.227 |
| 540 | 89.235 | 88.793 | 87.980 | 86.426 | 87.476 |
| 550 | 88.758 | 88.232 | 87.516 | 86.075 | 86.870 |
| 560 | 88.265 | 87.648 | 87.040 | 85.688 | 86.267 |
| 570 | 87.857 | 87.165 | 86.628 | 85.348 | 85.754 |
| 580 | 87.767 | 87.032 | 86.511 | 85.331 | 85.601 |
| 590 | 87.962 | 87.203 | 86.681 | 85.605 | 85.774 |
| 600 | 88.186 | 87.436 | 86.902 | 85.935 | 86.004 |
| 610 | 88.247 | 87.489 | 86.969 | 86.055 | 86.059 |
| 620 | 88.345 | 87.564 | 87.050 | 86.192 | 86.126 |
| 630 | 88.485 | 87.713 | 87.196 | 86.390 | 86.259 |
| 640 | 88.856 | 88.101 | 87.583 | 86.820 | 86.618 |
| 650 | 89.229 | 88.493 | 87.982 | 87.267 | 86.996 |
| 660 | 89.630 | 88.912 | 88.406 | 87.722 | 87.415 |
| 670 | 89.861 | 89.117 | 88.657 | 87.981 | 87.632 |
| 680 | 90.007 | 89.242 | 88.819 | 88.141 | 87.754 |
| 690 | 90.065 | 89.310 | 88.887 | 88.236 | 87.809 |
| 700 | 90.066 | 89.327 | 88.896 | 88.277 | 87.821 |
| 710 | 90.087 | 89.341 | 88.915 | 88.305 | 87.836 |
| 720 | 90.076 | 89.337 | 88.911 | 88.314 | 87.827 |
| 730 | 90.184 | 89.429 | 88.997 | 88.424 | 87.924 |
| 740 | 90.261 | 89.513 | 89.054 | 88.501 | 87.968 |
| 750 | 90.341 | 89.682 | 89.110 | 88.600 | 87.987 |

TABLE 13A
Reflectance of Example 5A and Example 5A Control

| | (Measured through | | (Measured through | |
|---|---|---|---|---|
| | Nylon Side) | | Polyester side) | |
| | | Example 5A | | Example 5A |
| Wavelength | Example 5A | Control | Example 5A | Control |
| (nm) | (14.4%) | (0%) | (14.4%) | (0%) |
| 360 | 10.80 | 7.43 | 21.37 | 18.89 |
| 370 | 9.041 | 6.082 | 24.638 | 22.082 |
| 380 | 8.722 | 5.753 | 26.246 | 23.578 |
| 390 | 11.341 | 7.747 | 28.274 | 25.351 |
| 400 | 24.935 | 18.686 | 36.268 | 32.439 |
| 410 | 57.559 | 47.297 | 56.612 | 50.811 |
| 420 | 95.044 | 86.489 | 83.974 | 77.617 |
| 430 | 119.201 | 116.550 | 104.485 | 101.018 |
| 440 | 122.047 | 122.348 | 108.518 | 107.630 |
| 450 | 115.143 | 115.889 | 104.350 | 104.276 |
| 460 | 106.568 | 106.917 | 98.606 | 98.605 |
| 470 | 100.533 | 100.502 | 94.571 | 94.513 |
| 480 | 97.432 | 97.198 | 92.576 | 92.554 |
| 490 | 94.413 | 93.919 | 90.606 | 90.494 |
| 500 | 92.877 | 92.215 | 89.689 | 89.511 |
| 510 | 91.403 | 90.511 | 88.843 | 88.550 |
| 520 | 90.295 | 89.189 | 88.263 | 87.839 |
| 530 | 89.560 | 88.274 | 87.296 | 87.372 |
| 540 | 89.197 | 87.774 | 87.793 | 87.111 |
| 550 | 88.955 | 87.412 | 87.679 | 86.899 |
| 560 | 88.820 | 87.179 | 87.629 | 86.774 |
| 570 | 88.827 | 87.140 | 87.725 | 86.805 |
| 580 | 88.860 | 87.123 | 87.803 | 86.836 |
| 590 | 88.935 | 87.179 | 87.915 | 86.929 |
| 600 | 89.114 | 87.385 | 88.138 | 87.175 |
| 610 | 89.251 | 87.585 | 88.306 | 87.416 |
| 620 | 89.504 | 87.940 | 88.598 | 87.792 |
| 630 | 89.810 | 88.340 | 88.939 | 88.222 |
| 640 | 90.234 | 88.816 | 89.376 | 88.734 |
| 650 | 90.551 | 89.181 | 89.746 | 89.121 |
| 660 | 90.904 | 89.584 | 90.126 | 89.561 |
| 670 | 91.219 | 89.986 | 90.478 | 89.975 |
| 680 | 91.483 | 90.270 | 90.764 | 90.285 |
| 690 | 91.750 | 90.559 | 91.050 | 90.588 |
| 700 | 91.886 | 90.729 | 91.216 | 90.777 |
| 710 | 91.971 | 90.809 | 91.310 | 90.884 |
| 720 | 92.014 | 90.846 | 91.373 | 90.940 |
| 730 | 92.025 | 90.852 | 91.418 | 90.956 |
| 740 | 91.960 | 90.767 | 91.374 | 90.886 |
| 750 | 91.847 | 90.625 | 91.276 | 90.739 |

TABLE 13B
Reflectance of Example 5B and Example 5B Control

| | (Measured through | | (Measured through | |
|---|---|---|---|---|
| | Nylon side) | | Polyester side) | |
| | | Example 5B | | Example 5B |
| Wavelength | Example 5B | Control | Example 5B | Control |
| (nm) | (20.1%) | (0%) | (20.1%) | (0%) |
| 360 | 48.20 | 37.37 | 39.09 | 33.06 |
| 370 | 59.510 | 49.121 | 53.704 | 46.976 |
| 380 | 66.743 | 56.887 | 62.759 | 55.918 |
| 390 | 71.565 | 61.994 | 68.191 | 61.273 |
| 400 | 75.052 | 65.964 | 72.033 | 65.221 |
| 410 | 77.447 | 68.948 | 74.745 | 68.209 |
| 420 | 79.281 | 71.376 | 76.795 | 70.602 |
| 430 | 80.807 | 73.465 | 78.740 | 72.662 |
| 440 | 82.115 | 75.283 | 79.959 | 74.508 |
| 450 | 83.300 | 76.950 | 81.345 | 76.228 |
| 460 | 84.433 | 78.532 | 82.686 | 77.895 |
| 470 | 85.412 | 79.930 | 83.859 | 79.391 |
| 480 | 86.268 | 81.181 | 84.899 | 80.736 |
| 490 | 86.916 | 82.140 | 85.701 | 81.771 |
| 500 | 87.545 | 83.042 | 86.457 | 82.737 |
| 510 | 87.959 | 83.664 | 86.976 | 83.407 |
| 520 | 88.266 | 84.137 | 87.361 | 83.924 |
| 530 | 88.566 | 84.543 | 87.733 | 84.370 |
| 540 | 88.861 | 84.923 | 88.095 | 84.779 |
| 550 | 89.049 | 85.181 | 88.337 | 85.073 |
| 560 | 89.215 | 85.394 | 88.552 | 85.319 |
| 570 | 89.435 | 85.655 | 88.817 | 85.603 |
| 580 | 89.615 | 85.853 | 89.051 | 85.839 |
| 590 | 89.795 | 86.066 | 89.272 | 86.086 |
| 600 | 90.027 | 86.393 | 89.543 | 86.421 |
| 610 | 90.200 | 86.641 | 89.749 | 86.710 |
| 620 | 90.468 | 86.998 | 90.045 | 87.110 |
| 630 | 90.777 | 87.386 | 90.402 | 87.542 |
| 640 | 91.157 | 87.825 | 90.819 | 88.034 |
| 650 | 91.441 | 88.149 | 91.137 | 88.434 |
| 660 | 91.746 | 88.493 | 91.474 | 88.817 |
| 670 | 92.024 | 88.839 | 91.784 | 89.156 |
| 680 | 92.246 | 89.091 | 92.033 | 89.445 |
| 690 | 92.544 | 89.421 | 92.351 | 89.822 |
| 700 | 92.732 | 89.669 | 92.562 | 90.100 |
| 710 | 92.854 | 89.841 | 92.703 | 90.300 |
| 720 | 92.939 | 89.982 | 92.808 | 90.463 |
| 730 | 92.997 | 90.074 | 92.876 | 90.575 |
| 740 | 92.981 | 90.070 | 92.858 | 90.597 |
| 750 | 92.922 | 89.987 | 92.796 | 90.546 |

TABLE 14
Reflectance of Comparative Examples A-E

| | Comparative | Comparative | Comparative | Comparative | Comparative |
|---|---|---|---|---|---|
| Wavelength (nm) | Example A | Example B | Example C | Example D | Example E |
| 360 | 14.720 | 19.650 | 20.716 | 10.151 | 33.155 |
| 370 | 23.794 | 21.652 | 23.292 | 10.477 | 34.267 |
| 380 | 29.113 | 22.815 | 25.320 | 10.525 | 34.831 |
| 390 | 30.961 | 23.679 | 27.120 | 10.464 | 35.830 |
| 400 | 31.480 | 24.275 | 28.441 | 10.372 | 37.109 |
| 410 | 31.630 | 24.835 | 29.436 | 10.257 | 38.212 |
| 420 | 31.694 | 25.182 | 29.968 | 10.105 | 38.758 |
| 430 | 31.664 | 24.964 | 29.706 | 9.919 | 38.727 |
| 440 | 31.600 | 24.585 | 29.115 | 9.745 | 38.287 |
| 450 | 31.516 | 23.989 | 28.151 | 9.612 | 37.669 |
| 460 | 31.439 | 23.446 | 27.120 | 9.552 | 37.066 |
| 470 | 31.342 | 23.056 | 26.191 | 9.549 | 36.597 |
| 480 | 31.253 | 22.736 | 25.302 | 9.626 | 36.237 |
| 490 | 31.110 | 22.432 | 24.416 | 9.757 | 35.878 |
| 500 | 31.040 | 22.283 | 23.712 | 9.976 | 35.583 |
| 510 | 30.895 | 22.123 | 23.033 | 10.203 | 35.230 |
| 520 | 30.769 | 22.025 | 22.446 | 10.491 | 34.932 |
| 530 | 30.601 | 21.937 | 21.919 | 10.780 | 34.645 |
| 540 | 30.476 | 21.935 | 21.533 | 11.130 | 34.485 |
| 550 | 30.302 | 21.938 | 21.212 | 11.472 | 34.381 |
| 560 | 30.153 | 21.986 | 20.989 | 11.844 | 34.404 |
| 570 | 29.999 | 22.072 | 20.846 | 12.229 | 34.551 |
| 580 | 29.871 | 22.213 | 20.798 | 12.644 | 34.852 |
| 590 | 29.716 | 22.365 | 20.801 | 13.048 | 35.224 |
| 600 | 29.580 | 22.580 | 20.877 | 13.511 | 35.754 |
| 610 | 29.472 | 22.822 | 20.988 | 13.967 | 36.322 |
| 620 | 29.364 | 23.094 | 21.122 | 14.420 | 36.953 |
| 630 | 29.223 | 23.361 | 21.249 | 14.853 | 37.575 |
| 640 | 29.102 | 23.684 | 21.392 | 15.291 | 38.239 |
| 650 | 28.945 | 24.012 | 21.520 | 15.709 | 38.868 |
| 660 | 28.810 | 24.390 | 21.663 | 16.139 | 39.537 |
| 670 | 28.659 | 24.800 | 21.818 | 16.565 | 40.202 |
| 680 | 28.518 | 25.262 | 21.996 | 16.995 | 40.899 |
| 690 | 28.515 | 25.884 | 22.323 | 17.538 | 41.734 |
| 700 | 28.527 | 26.602 | 22.722 | 18.110 | 42.611 |
| 710 | 28.468 | 27.286 | 23.094 | 18.611 | 43.427 |
| 720 | 28.386 | 27.986 | 23.488 | 19.086 | 44.216 |
| 730 | 28.197 | 28.651 | 23.866 | 19.462 | 44.937 |
| 740 | 27.907 | 29.281 | 24.234 | 19.758 | 45.574 |
| 750 | 27.323 | 29.804 | 24.524 | 19.892 | 46.068 |

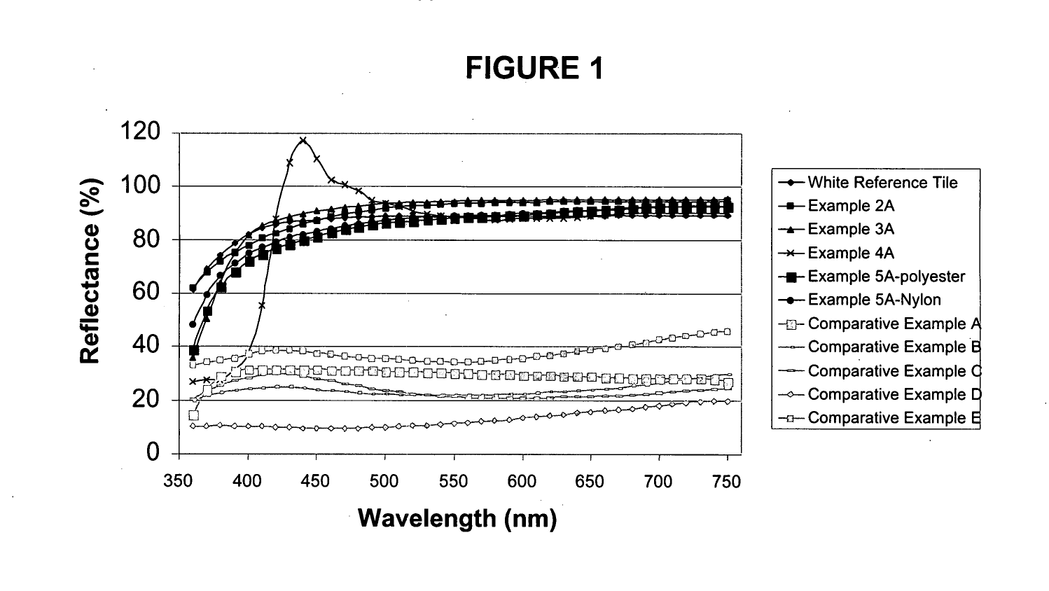

The reflectance data from Examples with the highest silver loadings (that is, Examples 2A, 3A, 4A, and 5B), along with the reflectance data from the Comparative Examples, is plotted in line graph in FIG. 1.

From the data, one observes that the present Examples exhibit significantly higher reflectance across the visible spectrum than do any of the Comparative Examples. These values are indicative of the white (highly reflective) nature of the present treated articles.

A review of FIG. 1 indicates that Examples 2A, 3A, 4A, and 5B are grouped fairly closely together on a line graph, all positioned around the plot of the reflectance data for a white reference tile, where wavelength (in nm) is the x axis and reflectance (%) is the y axis. This indicates that processing conditions have been established to ensure a treated article with a white surface, regardless of the amount of silver-based antimicrobial applied. Even with significant amounts of silver antimicrobial, such as the 20.7% of Example 2A and the 20.1% of Example 5B, the color of the treated article is not adversely affected.

The peak in reflectance values that is observed at wavelengths of between 400 nm and 500 nm in Example 4A is due to the presence of an optical brightener. Such brighteners are known to further enhance the reflectivity of an article.

### TEST 4: Whiteness/Yellowness Evaluation

Whiteness is measured using the Stensby Index for Whiteness. Higher values indicate a fabric with greater whiteness.

A correlating measure is ASTM E313-73 (D1925), which measures the yellowness of a sample. Higher values indicate a sample with more yellowness; negative values indicate a sample with less yellowness.

The Example fabrics (2-5) were measured, as described, with comparison being made to a white reference tile. The white ceramic tile was calibrated and traceable to the National Institute of Standards & Technology (NIST) and is referenced by the (NIST) report, standard reference material 2020c Serial number 27073, 844/259504-98R dated May 14, 1999. The Ceramic Standards are calibrated to the GretagMacbeth® Virtual database (STF19 Sphere). Calibration values displayed are CIELAB 1976, Illuminant D65, and 10° Observer at a controlled temperature of between 71° F. and 73° F.

The tests were run using GretagMacbeth Color Eye 7000A spectrophotometer, including a xenon flash light source, with results being provided in TABLES 15A and 15B.

TABLE 15A
Whiteness/Yellowness Measurements: Examples 2-4

| | Whiteness | Yellowness |
|---|---|---|
| Sample Identification | (Stensby Index) | (ASTM E313-73 (D1925)) |
| White Reference Tile | 89.317 | 9.210 |
| Example 2A | 80.622 | 14.233 |
| Example 2B | 77.545 | 15.456 |
| Example 2C | 82.669 | 13.260 |
| Example 2D | 75.450 | 16.479 |
| Example 2 Control | 81.702 | 13.735 |
| Example 3A | 87.117 | 11.713 |
| Example 3B | 88.961 | 11.040 |
| Example 3 Control | 89.985 | 10.636 |
| Example 4A | 128.443 | −7.284 |
| Example 4B | 135.182 | −10.349 |
| Example 4C | 126.565 | −6.920 |
| Example 4D | 131.201 | −8.165 |
| Example 4 Control | 136.404 | −11.474 |

TABLE 15B
Whiteness/Yellowness Measurements: Examples 5A and 5B

| | | | Yellowness |
|---|---|---|---|
| | Optical | Whiteness | (ASTM E313-73 |
| Sample Identification | Brightener | (Stensby Index) | (D1925)) |
| White Reference Tile | n/a | 85.799 | 11.812 |
| Example 5A | Yes | 142.318 | −9.885 |
| (nylon side) | | | |
| Example 5A Control | Yes | 141.381 | −10.773 |
| (nylon side) | | | |
| Example 5A | Yes | 121.151 | −2.038 |
| (polyester side) | | | |
| Example 5A Control | Yes | 118.114 | −1.807 |
| (polyester side) | | | |
| Example 5B | No | 80.489 | 14.295 |
| (nylon side) | | | |
| Example 5B Control | No | 71.776 | 17.427 |
| (nylon side) | | | |
| Example 5B | No | 77.268 | 15.762 |
| (polyester side) | | | |
| Example 5B Control | No | 70.546 | 18.172 |
| (polyester side) | | | |

The data in TABLES 15A and 15B indicates that, generally, the silver loading does not significantly affect the color of the device. In other words, a manufacturer can apply compositions containing various amounts of silver (and specifically, ALPHASAN® antimicrobial) to a substrate and still maintain a white color in the treated substrate. Heretofore, with other known silver compounds, it has been impossible to obtain a white treated substrate with the effective amounts of silver necessary for wound treatment and infection prevention.

### TEST 5: Color Evaluation: Lightness/Darkness, Yellow/Blue, Red/Green

Often, the surface color of an article is quantified using a series of measurements (L*, a*, and b*) generated by measuring the samples using a spectrophotometer. The equipment used for this test was a GretagMacbeth Color Eye 7000A spectrophotometer. The software program used was “Color imatch.” “L” is a measure of the amount of white or black in a sample; higher “L” values indicate a lighter colored sample. “A” is a measure of the amount of red or green in a sample, while “B” is a measure of the amount of blue or yellow in a sample.

Other measures made using the same testing equipment include C* and h°. C*, chroma, is a measure of the color saturation of the article. h°, hue, is a measure of the shade of the article.

TABLES 16A and 16B show a comparison of various samples, as tested by a GretagMacbeth Color Eye 7000A spectrophotometer using the white reference tile described in Test 4 as a standard. It is important to note that the values obtained are dependent upon the type of simulated light source used (e.g., incandescent, fluorescent, etc.) and the angle of observation. In these tests, a D65-10 setting was used (which represents daylight conditions), 6500° K. is the correlated color temperature, and 10° is the angle of observation.

TABLE 16A
Color Measurements of Examples 2-4 and Comparative Examples

| Sample ID | | | | | | |
|---|---|---|---|---|---|---|
| (% ALPHASAN ® | Sample | | | | | |
| Content) | Color | L* | C* | h° | a* | b* |
| White reference tile | White | 95.587 | 1.235 | 114.481 | −0.512 | 1.124 |
| Example 2A (20.7%) | White | 97.332 | 4.382 | 102.328 | −0.936 | 4.281 |
| Example 2B (8.2%) | White | 96.611 | 5.091 | 100.910 | −0.964 | 4.999 |
| Example 2C (4.2%) | White | 96.433 | 3.599 | 100.175 | −0.636 | 3.542 |
| Example 2D (1.1%) | White | 96.079 | 5.636 | 99.216 | −0.903 | 5.563 |
| Ex. 2 Control (0%) | White | 96.255 | 3.860 | 99.244 | −0.620 | 3.810 |
| Example 3A (18.2%) | White | 97.808 | 2.721 | 102.936 | −0.609 | 2.652 |
| Example 3B (2.2%) | White | 96.668 | 2.046 | 94.481 | −0.160 | 2.040 |
| Ex. 3 Control (0%) | White | 98.248 | 2.051 | 104.613 | −0.517 | 1.985 |
| Example 4A (16.0%) | White | 96.174 | 8.887 | 280.515 | 1.622 | −8.738 |
| Example 4B (9.9%) | White | 96.060 | 10.626 | 280.689 | 1.971 | −10.441 |
| Example 4C (5.0%) | White | 95.618 | 8.621 | 280.348 | 1.549 | −8.481 |
| Example 4D (1.2%) | White | 95.082 | 9.658 | 283.814 | 2.306 | −9.379 |
| Ex. 4 Control (0%) | White | 95.534 | 11.162 | 280.374 | 2.010 | −10.980 |
| Comparative Ex. A | Black interior; white | 61.900 | 1.898 | 245.779 | −0.779 | −1.731 |
| | outer layers | | | | | |
| Comparative Ex. B | Dark bluish-gray | 54.396 | 3.595 | 310.716 | 2.345 | −2.725 |
| | metallic | | | | | |
| Comparative Ex. C | Dark bluish-gray | 54.025 | 9.870 | 281.791 | 2.017 | −9.662 |
| | metallic | | | | | |
| Comparative Ex. D | Dark gray | 40.804 | 7.903 | 48.557 | 5.231 | 5.925 |
| Comparative Ex. E | Light blue-gray | 65.994 | 3.927 | 313.587 | 2.708 | −2.845 |

TABLE 16B
Color Measurements of Examples 5 and 6

| Sample ID | | | | | | |
|---|---|---|---|---|---|---|
| (Side on which | % | | | | | |
| measurement was | ALPHASAN ® | | | | | |
| made) | Content | L* | C* | h° | a* | b* |
| White reference tile | n/a | 95.513 | 2.523 | 95.953 | −0.262 | 2.509 |
| Example 5A | 14.4% | 96.332 | 11.471 | 288.519 | 3.643 | −10.877 |
| (nylon side) | | | | | | |
| Example 5A Control | 0% | 95.779 | 11.627 | 286.509 | 3.304 | −11.147 |
| (nylon side) | | | | | | |
| Example 5A | 14.4% | 95.518 | 6.618 | 290.143 | 2.279 | −6.214 |
| (polyester side) | | | | | | |
| Example 5A Control | 0% | 95.223 | 6.142 | 286.884 | 1.784 | −5.878 |
| (polyester side) | | | | | | |
| Example 5B | 20.1% | 95.489 | 4.081 | 96.754 | −0.480 | 4.053 |
| (nylon side) | | | | | | |
| Example 5B Control | 0% | 93.771 | 6.037 | 97.857 | −0.825 | 5.981 |
| (nylon side) | | | | | | |
| Example 5B | 20.1% | 95.162 | 4.969 | 96.393 | −0.553 | 4.938 |
| (polyester side) | | | | | | |
| Example 5B Control | 0% | 93.716 | 6.460 | 97.150 | −0.804 | 6.410 |
| (polyester side) | | | | | | |
| Example 6 | n/a | 67.814 | 11.041 | 291.904 | 4.119 | −10.244 |
| (nylon side) | | | | | | |
| Example 6 | n/a | 68.382 | 4.214 | 291.399 | 1.537 | −3.923 |
| (polyester side) | | | | | | |

Looking at the L* values in TABLES 16A and 16B for Examples 2-6, it is apparent that there is a slight reduction in L* values as the amount of silver-containing antimicrobial agent (ALPHASAN®) is decreased. As noted, Examples 4A-4D and 5A contain an optical brightening agent, which causes the Examples to emit blue light and which results in the higher h° values shown in TABLES 16A and 16B. TABLE 16A shows that the Comparative Examples each have an L* value of less than 66.0, indicating the presence of darker shades of color.

One contemplated benefit of having an antimicrobial wound care device whose color has not been altered by the addition of a silver antimicrobial agent is that the wound care device can be dyed a number of different colors. For example, the wound care devices could be dyed to represent different end uses or levels of antimicrobial agent. Representative colored wound care substrates were prepared as described above. Examples 7A-7F and the corresponding Control samples were measured using the color evaluation techniques and equipment described previously. The results are shown in TABLES 17A and 17B.

TABLE 17A
Color Evaluation: Examples 7A-7F and Corresponding Control Samples
(measured through the nylon side of the fabric with D-65 light source)

| Sample ID | Sample Color | L* | a* | b* | C* | h° | DE CMC |
|---|---|---|---|---|---|---|---|
| Reference tile | white | 95.518 | −0.278 | 2.498 | 2.513 | 96.358 | n/a |
| Example 7A | blue | 75.609 | −11.036 | −13.713 | 17.602 | 231.173 | 26.006 |
| Control 7A | blue | 72.076 | −10.778 | −14.562 | 18.116 | 233.493 | 27.052 |
| Example 7B | blue | 82.515 | −8.789 | −9.976 | 13.295 | 228.618 | 20.077 |
| Control 7B | blue | 77.970 | −9.585 | −11.618 | 15.061 | 230.475 | 22.682 |
| Example 7C | green | 84.542 | −11.911 | 21.160 | 24.282 | 119.375 | 28.010 |
| Control 7C | green | 81.022 | −13.322 | 23.755 | 27.236 | 119.285 | 31.864 |
| Example 7D | green | 88.135 | −9.642 | 18.373 | 20.750 | 117.690 | 23.402 |
| Control 7D | green | 85.300 | −10.861 | 20.531 | 23.227 | 117.880 | 26.620 |
| Example 7E | purple | 74.010 | 7.616 | −12.837 | 14.541 | 301.587 | 23.055 |
| Control 7E | purple | 68.487 | 7.725 | −14.171 | 16.139 | 298.595 | 25.660 |
| Example 7F | purple | 80.969 | 5.585 | −8.701 | 10.340 | 302.697 | 17.240 |
| Control 7F | purple | 75.808 | 6.527 | −10.181 | 12.094 | 302.666 | 19.907 |

TABLE 17B
Color Evaluation: Examples 7A-7F and Corresponding Control Samples
(measured through the polyester side of the fabric with D-65 light source)

| Sample ID | Sample Color | L* | a* | b* | C* | h° | DE CMC |
|---|---|---|---|---|---|---|---|
| Reference tile | white | 95.518 | −0.278 | 2.498 | 2.513 | 96.358 | n/a |
| Example 7A | blue | 82.818 | −5.967 | −7.422 | 9.523 | 231.201 | 15.557 |
| Control 7A | blue | 78.653 | −5.948 | −8.324 | 10.231 | 234.454 | 16.953 |
| Example 7B | blue | 85.706 | −5.434 | −5.993 | 8.090 | 227.803 | 13.444 |
| Control 7B | blue | 81.367 | −6.069 | −7.245 | 9.451 | 230.050 | 15.578 |
| Example 7C | green | 87.336 | −7.057 | 12.500 | 14.354 | 119.447 | 15.523 |
| Control 7C | green | 84.051 | −8.241 | 13.740 | 16.008 | 120.872 | 17.827 |
| Example 7D | green | 89.884 | −5.869 | 10.917 | 12.395 | 118.262 | 12.919 |
| Control 7D | green | 87.047 | −7.015 | 12.529 | 14.359 | 119.247 | 15.542 |
| Example 7E | purple | 82.736 | 3.165 | −6.167 | 6.932 | 297.169 | 13.036 |
| Control 7E | purple | 77.424 | 3.566 | −7.519 | 8.321 | 295.375 | 15.383 |
| Example 7F | purple | 85.972 | 3.027 | −4.732 | 5.617 | 302.611 | 11.006 |
| Control 7F | purple | 80.853 | 3.706 | −6.258 | 7.272 | 300.633 | 13.615 |

The results shown in TABLES 17A and 17B indicate that the application of the antimicrobial finish described herein does not adversely alter the overall color of the wound care substrate (that is, the DE CMC values are substantially the same between each Example and its Control).

Further, the Example fabrics exhibited an L* value of at least 74.0, a relatively high level of lightness.

### TEST 6: DE CMC Evaluation

Yet another measurement of the relative color of the samples is DE CMC. DE CMC is a measure of the overall color difference for all uniform color spaces, where DE CMC represents the magnitude of difference between a color and a reference (in this case, a pure white standard). The higher the DE CMC value, the more pronounced the difference in color. Said another way, smaller DE CMC values represent colors that are closer to white.

The GretagMacbeth Color Eye 7000A Spectrophotometer calculates DE CMC values based on wavelength and reflectance data for each sample. Examples 2A-2D, 3A-3B, 4A-4D, and 5A-5B were compared to the Comparative Examples. The results are shown in TABLE 18.

TABLE 18
DE CMC: Comparison of Magnitude of Color Difference

| Sample Identification | L* value | |
|---|---|---|
| (% active antimicrobial) | (Lightness of sample) | DE CMC |
| Example 2A (20.7%) | 97.332 | 4.493 |
| Example 2B (8.2%) | 96.611 | 5.465 |
| Example 2C (4.2%) | 96.433 | 3.398 |
| Example 2D (1.1%) | 96.079 | 6.231 |
| Example 2 Control (0%) | 96.255 | 3.765 |
| Example 3A (18.2%) | 97.808 | 2.273 |
| Example 3B (2.2%) | 96.668 | 1.425 |
| Example 3 Control (0%) | 98.248 | 1.511 |
| Example 4A (16.0%) | 96.174 | 14.166 |
| Example 4B (9.9%) | 96.060 | 16.596 |
| Example 4C (5.0%) | 95.618 | 13.791 |
| Example 4D (1.2%) | 95.082 | 15.262 |
| Example 4 Control (0%) | 95.534 | 17.343 |
| Example 5A (14.4%; nylon side) | 96.332 | 18.171 |
| Example 5A Control (0%; nylon side) | 95.779 | 18.387 |
| Example 5A (14.4%; polyester side) | 95.518 | 11.974 |
| Example 5A Control (0%; polyester | 95.223 | 11.395 |
| side) | | |
| Example 5B (20.1%; nylon side) | 95.489 | 1.964 |
| Example 5B Control (0%; nylon side) | 93.771 | 4.472 |
| Example 5B (20.1%; polyester side) | 95.162 | 3.084 |
| Example 5B Control (0%; polyester | 93.716 | 5.000 |
| side) | | |
| Comparative Example A | 61.900 | 12.244 |
| Comparative Example B | 54.396 | 15.661 |
| Comparative Example C | 54.025 | 21.094 |
| Comparative Example D | 40.804 | 21.519 |
| Comparative Example E | 65.994 | 12.443 |

The results shown in TABLE 18 indicate that, whereas the experimental Examples without optical brighteners exhibited DE CMC value no higher than 6.2, all of the Comparative Examples exhibited a DE CMC value higher than 12. This finding indicates that the Examples prepared in accordance with the teachings herein exhibited less difference from the white standard than did the Comparative Examples.

### TEST 7: Color Stability Testing

It is desirable that the color of the antimicrobial wound care device does not change significantly with the passage of time. With traditional silver-based antimicrobial devices, exposure to light causes a color change in the form of browning or graying. The present device overcomes this shortcoming.

To evaluate the color durability of the present device, the yellowness of treated Example 2E fabric and untreated Example 2 Control fabric were measured periodically over a 26-day period, using the test method described previously. Both fabrics were subjected to cool-white fluorescent lighting for 24-hour continuous exposure. Five different areas of each sample were evaluated and averaged. The data is reported below in TABLE 19.

TABLE 19
Color Durability Testing using Yellowness Index Measure

| Days of Exposure | Control Fabric | Example 2E |
|---|---|---|
| 0 | 13.478 | 13.564 |
| 1 | 13.728 | 13.716 |
| 2 | 13.547 | 13.724 |
| 3 | 13.784 | 13.706 |
| 4 | 13.892 | 13.700 |
| 7 | 14.020 | 13.799 |
| 8 | 13.987 | 13.821 |
| 11 | 14.113 | 13.844 |
| 14 | 14.110 | 13.715 |
| 16 | 14.201 | 13.667 |
| 18 | 14.687 | 14.128 |
| 22 | 14.190 | 13.664 |
| 23 | 14.188 | 13.641 |
| 24 | 14.145 | 13.573 |
| 25 | 14.204 | 13.757 |

The results show that the Example 2E performed comparably with the untreated fabric in maintaining its original color over time. There were no visible signs of browning or graying, as are common with silver-containing articles that are exposed to the environment. Rather, Example 2E maintained its white appearance throughout the evaluation period.

As described previously, any of the substrates described herein may be used alone as a wound care device, including fabrics and alginates. Alternatively, one or more of these substrates may be joined together in any possible combination to form a composite, multi-layered, wound care device. The layers may be joined together through various techniques such as ultrasonic welding, heat or pressure lamination, the use of adhesives, needle punching, hydraulic needling, sewing, or other fiber and/or fabric layer laminating or joining processes known to those skilled in the art. The layers may be joined together only at intermittent locations or the layers may be joined together completely.

The topical antimicrobial finish of the current invention may be applied to any one or more of the substrate layers comprising the composite wound care device. Additionally, an odor absorbing agent or layer may be included on or within one or more layers of the composite wound care device. Furthermore, in some instances, the wound care device may have an adhesive layer so that the device may be held in place over the wound site. In such cases, a layer of removable film may be placed over the wound-facing side of the wound care device to protect the adhesive layer until ready for use. Alternatively, the wound care device may be held in place by wrapping long pieces of wound dressing, such as gauze, over and around the wound care device and securing the free end in place by any suitable means, such as tape, adhesive, pins, clips, or hooks.

Thus, the above description and examples show that a topical antimicrobial finish may be applied to a variety of substrates to achieve an antimicrobially effective, silver-containing wound care device having the desired characteristics of antimicrobial efficacy, functional release of silver, and absence of a dark color. As has been described herein, the present wound care device possesses a significant advantage over competitive products, in that it exhibits a white color uncharacteristic of silver-containing antimicrobial articles and in that the white color is sustainable over long periods (i.e., in production, transit, and storage).

These and other modifications and variations to the present invention may be practiced by those of ordinary skill in the art, without departing from the spirit and scope of the present invention. Furthermore, those of ordinary skill in the art will appreciate that the foregoing description is by way of example only, and is not intended to limit the scope of the invention described in the appended claims.

1. A wound care device having a substrate selected from the group consisting of fibers, fabrics, and alginates, at least a portion of said substrate being coated with a non-electrically conductive finish,
wherein said finish consists essentially of at least one binder material and at least one compound that releases silver ions,
wherein said coated wound care device exhibits an L* value, before use, of at least 74.0, when measured between wavelengths of 360 nm and 750 nm using a D65 light source and a 10° observation angle.

2. The wound care device of claim 1, wherein said at least one silver ion-releasing compound is selected from the group consisting of silver ion exchange materials, silver particles, silver nanoparticles, silver salts, silver glass, and mixtures thereof.

3. The wound care device of claim 2, wherein said silver ion exchange material is selected from the group consisting of silver zirconium phosphate, silver calcium phosphate, silver zeolite, and mixtures thereof.

4. The wound care device of claim 3, wherein said silver ion exchange material is silver zirconium phosphate.

5. The wound care device of claim 1, wherein said wound care device exhibits an L* value of at least 80.0.

6. The wound care device of claim 5, wherein said wound care device exhibits an L* value of at least 90.0.

7. The wound care device of claim 1, wherein said substrate onto which said finish is applied includes additives selected from the group consisting of optical brighteners, dyes, opacifiers, and pigments.

8. The wound care device of claim 7, wherein said substrate comprises optical brighteners.

9. The wound care device of claim 1, wherein said substrate onto which said finish is applied is a fabric.

10. The wound care device of claim 9, wherein said substrate is a woven fabric.

11. The wound care device of claim 9, wherein said substrate is a nonwoven fabric.

12. The wound care device of claim 9, wherein said substrate is a knit fabric.

13. The wound care device of claim 12, wherein said knit fabric is a circular knit fabric comprised of polyester, nylon, and spandex, such that said knit fabric has a polyester side and a nylon side.

14. The wound care device of claim 1, wherein said device exhibits effective antimicrobial properties, as defined by a zone of inhibition against bacteria of at least 1 mm after two successive exposures.
